(19) United States
(12) Patent Application Publication
(10) Pub. No.: US 2026/0226027 A1
(43) Pub. Date: Aug. 6, 2026

(54) DEUTERATED COMPOUNDS

(71) Applicants: Neurocrine Biosciences, Inc., San Diego, CA (US); Nxera Pharma UK Limited, Cambridge (GB)

(72) Inventors: Evan Smith, San Diego, CA (US); Giles Albert Brown, Cambridge (GB); Miles Stuart Congreve, Cambridge (GB)

(21) Appl. No.: 19/149,696

(22) PCT Filed: Jan. 19, 2024
(86) PCT No.: PCT/US2024/012146

§ 371 Date: Jul. 21, 2025

Related U.S. Application Data
(60) Provisional application No. 63/440,812, filed on Jan. 24, 2023.

(51) Int. Cl. C07D 401/14; A61K 31/454; C07B 59/00

(52) U.S. Cl. CPC C07D 401/14; A61K 31/454; C07B 59/002; C07B 2200/05

(57) ABSTRACT

The present disclosure provides deuterated compounds and their use as agonists of the M4 muscarinic acetylcholine receptor in the treatment or prevention of various diseases or disorders associated with the M4 receptor.

### TECHNICAL FIELD

The present invention relates to deuterated compounds and their use as selective agonists of the M₄ muscarinic acetylcholine receptor and which are useful in the treatment or prevention of various diseases or disorders associated with the M₄ receptor.

### BACKGROUND

Muscarinic acetylcholine receptors (mAChRs) are members of the G protein-coupled receptor superfamily which mediate the actions of the neurotransmitter acetylcholine in both the central and peripheral nervous system. Five mAChR subtypes have been cloned, M₁ to M₅. The M₁ mAChR is predominantly expressed post-synaptically in the cortex, hippocampus, striatum and thalamus; M₂ mAChRs are located predominantly in the brainstem and thalamus, though also in the cortex, hippocampus and striatum where they reside on cholinergic synaptic terminals (Langmead et al., 2008 Br J Pharmacol). However, M₂ mAChRs are also expressed peripherally on cardiac tissue (where they mediate the vagal innervation of the heart) and in smooth muscle and exocrine glands. M₃ mAChRs are expressed at relatively low level in the CNS but are widely expressed in smooth muscle and glandular tissues such as sweat and salivary glands (Langmead et al., 2008 Br J Pharmacol).

Muscarinic receptors in the central nervous system, especially the M₁ mAChR, play a critical role in mediating higher cognitive processing. Diseases associated with cognitive impairments, such as Alzheimer's disease, are accompanied by loss of cholinergic neurons in the basal forebrain (Whitehouse et al., 1982 Science). In schizophrenia, which also has cognitive impairment as an important component of the clinical picture, mAChR density is reduced in the pre-frontal cortex, hippocampus and caudate putamen of schizophrenic subjects (Dean et al., 2002 Mol Psychiatry). Furthermore, in animal models, blockade or damage to central cholinergic pathways results in profound cognitive deficits and non-selective mAChR antagonists have been shown to induce psychotomimetic effects in psychiatric patients. Cholinergic replacement therapy has largely been based on the use of acetylcholinesterase inhibitors to prevent the breakdown of endogenous acetylcholine. These compounds have shown efficacy versus symptomatic cognitive decline in the clinic, but give rise to dose-limiting adverse events resulting from stimulation of peripheral M₂ and M₃ mAChRs including disturbed gastrointestinal motility, bradycardia, nausea and vomiting. Muscarinic receptors have also been implicated in the neurobiology of addiction. The reinforcing effects of cocaine and other addictive substances are mediated by the mesolimbic dopamine system where behavioral and neurochemical studies have shown that the cholinergic muscarinic receptor subtypes play important roles in regulation of dopaminergic neurotransmission. For example, M₄ (−/−) mice demonstrated significantly enhanced reward driven behaviour as result of exposure to cocaine (Schmidt et al., Psychopharmacology, 2011 August; 216(3):367-78). Furthermore, xanomeline has been demonstrated to block the effects of cocaine in these models.

### SUMMARY

The present application provides, inter alia, a compound of Formula I:

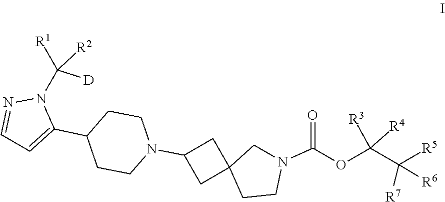

or a pharmaceutically acceptable salt thereof, wherein the constituent members are defined herein.

The present invention further provides pharmaceutical compositions comprising a compound described herein, or a pharmaceutically acceptable salt thereof, and a pharmaceutically acceptable carrier.

The present invention further provides methods of agonizing a muscarinic M₄ receptor, comprising contacting the M₄ receptor with a compound described herein, or a pharmaceutically acceptable salt thereof.

The present invention further provides methods of agonizing a muscarinic M₄ receptor in a patient, comprising contacting the M₄ receptor with a compound described herein, or a pharmaceutically acceptable salt thereof.

The present invention further provides methods of treating a disease or disorder associated with a muscarinic M₄ receptor in a patient, comprising administering to the patient a compound described herein, or a pharmaceutically acceptable salt thereof.

The present invention further provides compounds described herein, or a pharmaceutically acceptable salt thereof, for use in any of the methods described herein.

The present invention further provides uses of a compound described herein, or a pharmaceutically acceptable salt thereof, for the preparation of a medicament for use in any of the methods described herein.

### DESCRIPTION OF DRAWINGS

FIG. 1 shows proposed metabolic pathways for the observed Compound 1 metabolites from the in vitro studies described in Example 1.

FIG. 2 shows proposed metabolites of Compound 1 detected in human plasma and urine from the studies described in Example 2.

### DETAILED DESCRIPTION

Compound 1 (i.e., cis ethyl 2-[4-(1-methyl-1H-pyrazol-5-yl)piperidin-1-yl]-6-azaspiro[3.4]octane-6-carboxylate) is a muscarinic receptor agonist that is selective for the M4 muscarinic acetylcholine receptor (e.g., over any of M1, M2, and/or M3 receptors; see e.g., U.S. Pat. Nos. 9,670,183; 9,926,297; 10,196,380; 10,385,039; 10,689,368; and 10,961,225; the disclosures of which are each incorporated herein by reference in their entireties).

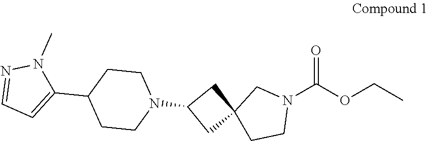 [SMILES: CCOC(=O)N1CC[C@]2(C1)C[C@@H](N1CCC(c3ccnn3C)CC1)C2]

The present application provides deuterated analogs of Compound 1, and pharmaceutically acceptable salts thereof. Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution of hydrogen at one or more metabolism sites with deuterium may afford one or more of the therapeutic advantages.

In some embodiments, the present application provides a compound of Formula I:

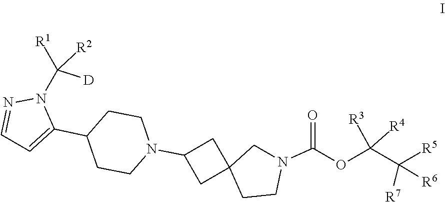

or a pharmaceutically acceptable salt thereof,
wherein R¹, R², R³, R⁴, R⁵, R⁶, and R⁷ are each independently selected from hydrogen and deuterium.

In some embodiments, zero to seven of R¹, R², R³, R⁴, R⁵, R⁶, and R⁷ are deuterium. In some embodiments, one to seven of R¹, R², R³, R⁴, R⁵, R⁶, and R⁷ are deuterium. In some embodiments, two to seven of R¹, R², R³, R⁴, R⁵, R⁶, and R⁷ are deuterium. In some embodiments, three to seven of R¹, R², R³, R⁴, R⁵, R⁶, and R⁷ are deuterium. In some embodiments, four to seven of R¹, R², R³, R⁴, R⁵, R⁶, and R⁷ are deuterium. In some embodiments, five to seven of R¹, R², R³, R⁴, R⁵, R⁶, and R⁷ are deuterium. In some embodiments, six to seven of R¹, R², R³, R⁴, R⁵, R⁶, and R⁷ are deuterium.

In some embodiments, zero to six of R¹, R², R³, R⁴, R⁵, R⁶, and R⁷ are deuterium. In some embodiments, one to six of R¹, R², R³, R⁴, R⁵, R⁶, and R⁷ are deuterium. In some embodiments, two to six of R¹, R², R³, R⁴, R⁵, R⁶, and R⁷ are deuterium. In some embodiments, three to six of R¹, R², R³, R⁴, R⁵, R⁶, and R⁷ are deuterium. In some embodiments, four to six of R¹, R², R³, R⁴, R⁵, R⁶, and R⁷ are deuterium. In some embodiments, five to six of R¹, R², R³, R⁴, R⁵, R⁶, and R⁷ are deuterium.

In some embodiments, zero to five of R¹, R², R³, R⁴, R⁵, R⁶, and R⁷ are deuterium. In some embodiments, one to five of R¹, R², R³, R⁴, R⁵, R⁶, and R⁷ are deuterium. In some embodiments, two to five of R¹, R², R³, R⁴, R⁵, R⁶, and R⁷ are deuterium. In some embodiments, three to five of R¹, R², R³, R⁴, R⁵, R⁶, and R⁷ are deuterium. In some embodiments, four to five of R¹, R², R³, R⁴, R⁵, R⁶, and R⁷ are deuterium.

In some embodiments, zero to four of R¹, R², R³, R⁴, R⁵, R⁶, and R⁷ are deuterium. In some embodiments, one to four of R¹, R², R³, R⁴, R⁵, R⁶, and R⁷ are deuterium. In some embodiments, two to four of R¹, R², R³, R⁴, R⁵, R⁶, and R⁷ are deuterium. In some embodiments, three to four of R¹, R², R³, R⁴, R⁵, R⁶, and R⁷ are deuterium.

In some embodiments, one of R¹, R², R³, R⁴, R⁵, R⁶, and R⁷ is deuterium. In some embodiments, two of R¹, R², R³, R⁴, R⁵, R⁶, and R⁷ are each deuterium. In some embodiments, three of R¹, R², R³, R⁴, R⁵, R⁶, and R⁷ are each deuterium. In some embodiments, four of R¹, R², R³, R⁴, R⁵, R⁶, and R⁷ are each deuterium. In some embodiments, five of R¹, R², R³, R⁴, R⁵, R⁶, and R⁷ are each deuterium. In some embodiments, six of R¹, R², R³, R⁴, R⁵, R⁶, and R⁷ are each deuterium. In some embodiments, R¹, R², R³, R⁴, R⁵, R⁶, and R⁷ are each deuterium.

In some embodiments, the compound of Formula I is a compound of Formula II:

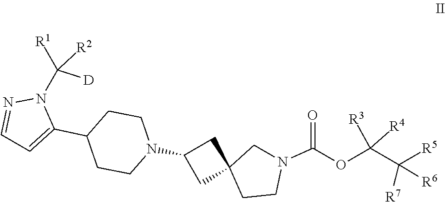

or a pharmaceutically acceptable salt thereof.

In some embodiments, the compound of Formula I is a compound of Formula III:

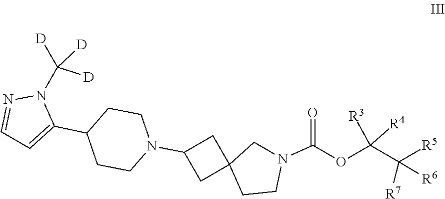

or a pharmaceutically acceptable salt thereof.

In some embodiments, the compound of Formula I is a compound of Formula IIIa:

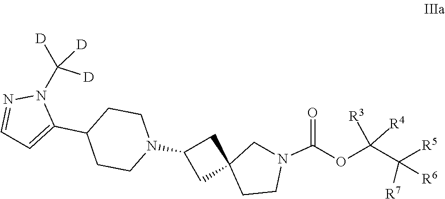

or a pharmaceutically acceptable salt thereof.

In some embodiments, the compound of Formula I is a compound of Formula IV:

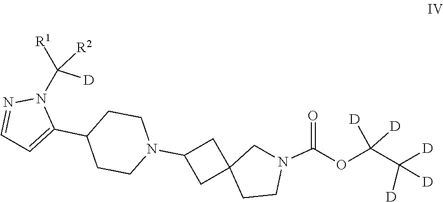

or a pharmaceutically acceptable salt thereof.

In some embodiments, the compound of Formula I is a compound of Formula IVa:

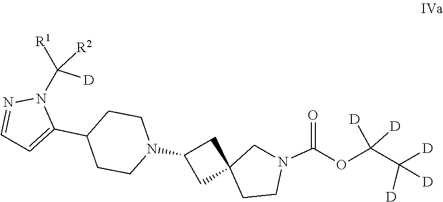

or a pharmaceutically acceptable salt thereof.

In some embodiments, the compound provided herein is selected from:

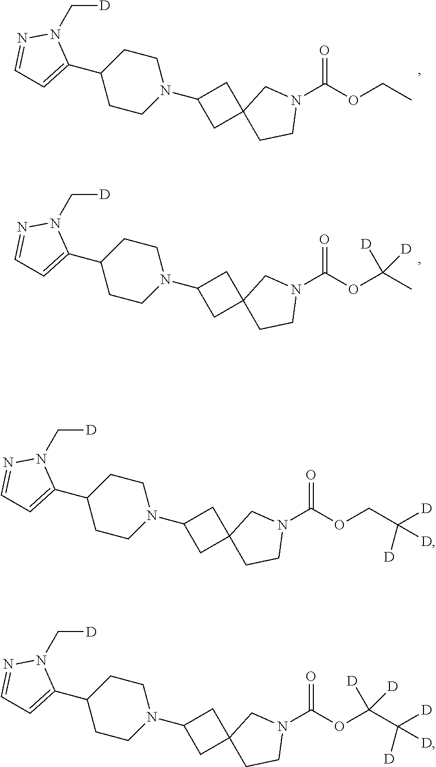

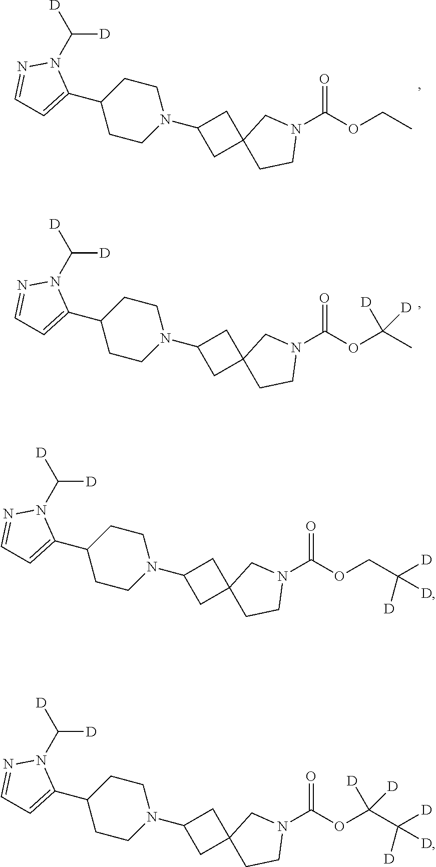

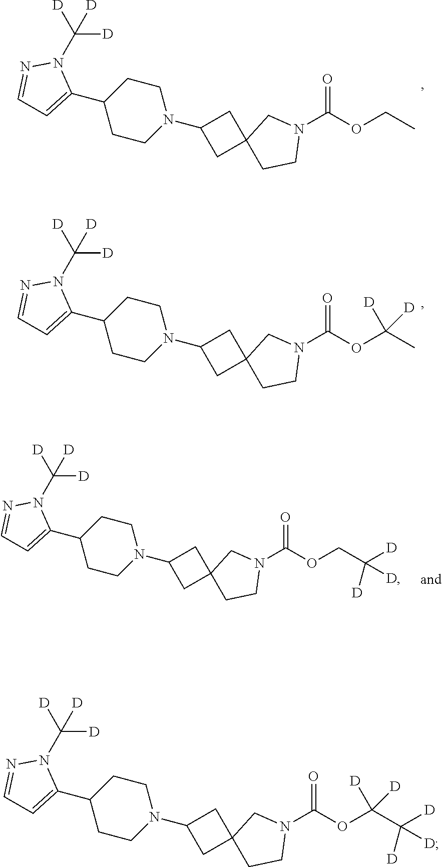

or a pharmaceutically acceptable salt thereof.

In some embodiments, the compound provided herein is selected from:

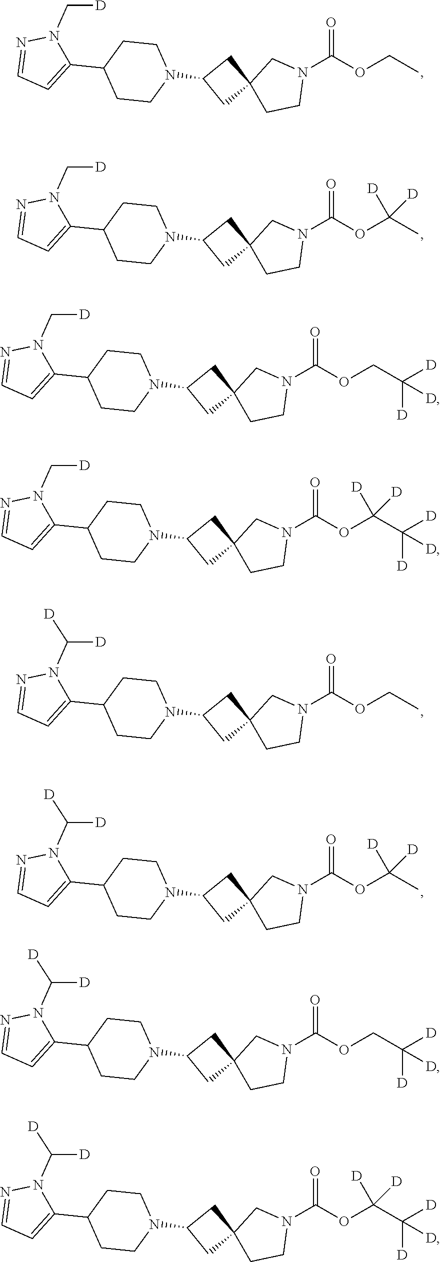

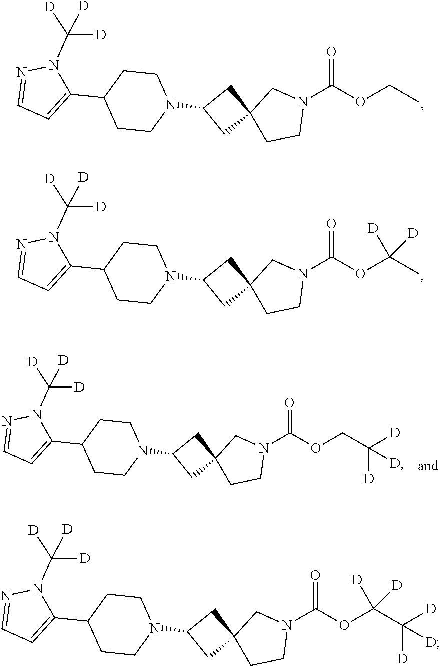

or a pharmaceutically acceptable salt thereof.

The compounds disclosed and described herein allow atoms at each position of the compound independently to have: 1) an isotopic distribution for a chemical element in proportional amounts to those usually found in nature or 2) an isotopic distribution in proportional amounts different to those usually found in nature unless the context clearly dictates otherwise. A particular chemical element has an atomic number defined by the number of protons within the atom's nucleus. Each atomic number identifies a specific element, but not the isotope; an atom of a given element may have a wide range in its number of neutrons. The number of both protons and neutrons in the nucleus is the atom's mass number, and each isotope of a given element has a different mass number. A compound wherein one or more atoms have an isotopic distribution for a chemical element in proportional amounts different to those usually found in nature is commonly referred to as being an isotopically-labeled compound. Each chemical element as represented in a compound structure may include any isotopic distribution of said element. For example, in a compound structure a hydrogen atom may be explicitly disclosed or understood to be present in the compound. At any position of the compound that a hydrogen atom may be present, the hydrogen atom can be an isotopic distribution of hydrogen, including but not limited to protium (¹H) and deuterium (²H) in proportional amounts to those usually found in nature and in proportional amounts different to those usually found in nature. Thus, reference herein to a compound encompasses all potential isotopic distributions for each atom unless the context clearly dictates otherwise. Examples of isotopes include isotopes of hydrogen, carbon, nitrogen, oxygen, phosphorous, sulfur, fluorine, chlorine, bromine, and iodine. As one of skill in the art would appreciate, any of the compounds as disclosed and described herein may include radioactive isotopes. Accordingly, also contemplated is use of compounds as disclosed and described herein, wherein one or more atoms have an isotopic distribution different to those usually found in nature, such as having ²H or ³H in greater proportion, or ¹¹C, ¹³C, or ¹⁴C in greater proportion than found in nature. By way of general example, and without limitation, isotopes of hydrogen include protium (¹H), deuterium (²H), and tritium (³H). Isotopes of carbon include carbon-11 (¹¹C), carbon-12 (¹²C), carbon-13 (¹³C), and carbon-14 (¹⁴C). Isotopes of nitrogen include nitrogen-13 (¹³N), nitrogen-14 (¹⁴N) and nitrogen-15 (¹⁵N). Isotopes of oxygen include oxygen-14 (¹⁴O), oxygen-15 (¹⁵O), oxygen-16 (¹⁶O), oxygen-17 (¹⁷O), and oxygen-18 (¹⁸O). Isotope of fluorine include fluorine-17 (¹⁷F), fluorine-18 (¹⁸F) and fluorine-19 (¹⁹F). Isotopes of phosphorous include phosphorus-31 (³¹P), phosphorus-32 (³²P), phosphorus-33 (³³P), phosphorus-34 (³⁴P), phosphorus-35 (³⁵P) and phosphorus-36 (³⁶P). Isotopes of sulfur include sulfur-32 (³²S), sulfur-33 (³³S), sulfur-34 (³⁴S), sulfur-35 (³⁵S), sulfur-36 (³⁶S) and sulfur-38 (³⁸S). Isotopes of chlorine include chlorine-35 (³⁵Cl), chlorine-36 (³⁶Cl) and chlorine-37 (³⁷Cl). Isotopes of bromine include bromine-75 (⁷⁵Br), bromine-76 (⁷⁶Br), bromine-77 (⁷⁷Br), bromine-79 (⁷⁹Br), bromine-81 (⁸¹Br) and bromine-82 (⁸²Br). Isotopes of iodine include iodine-123 (¹²³I), iodine-124 (¹²⁴I), iodine-125 (¹²⁵I), iodine-131 (¹³¹I) and iodine-135 (¹³⁵I). In some embodiments, atoms at every position of the compound have an isotopic distribution for each chemical element in proportional amounts to those usually found in nature. In some embodiments, an atom in one position of the compound has an isotopic distribution for a chemical element in proportional amounts different to those usually found in nature (remainder atoms having an isotopic distribution for a chemical element in proportional amounts to those usually found in nature). In some embodiments, atoms in at least two positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least three positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least four positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least five positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least six positions of the compound independently have an isotopic distribution for a chemical element in proportional amounts different to those usually found in nature (remainder atoms having an isotopic distribution for a chemical element in proportional amounts to those usually found in nature).

Certain compounds, for example those having incorporated radioactive isotopes such as ³H and ¹⁴C, are also useful in drug or substrate tissue distribution assays. Tritium (³H) and carbon-14 (¹⁴C) isotopes are particularly preferred for their ease of preparation and detectability. Compounds with isotopes such as deuterium (2H) in proportional amounts greater than usually found in nature may afford certain therapeutic advantages resulting from greater metabolic stability, such as, for example, increased in vivo half-life or reduced dosage requirements. Isotopically-labeled compounds can generally be prepared by performing procedures routinely practiced in the chemical art. Methods are readily available to measure such isotope perturbations or enrichments, such as, mass spectrometry, and for isotopes that are radio-isotopes additional methods are available, such as, radio-detectors used in connection with HPLC or GC.

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, protium (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound of the invention contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can include ²H as the major isotopic form, as example, or any carbon include be ¹³C as the major isotopic form, as example, or any nitrogen can include ¹⁵N as the major isotopic form, as example, and any oxygen can include ¹⁸O as the major isotopic form, as example. In certain embodiments, an “isotopic variant” of a compound contains an unnatural proportion of deuterium (²H).

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

The present disclosure further provides synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

### Methods of Use

The compounds of the present invention have activity as selective muscarinic M4 receptor agonists (e.g., selective over any of M₁, M₂, and/or M₃). The muscarinic activity of Compound 1 was previously reported, e.g., in U.S. Pat. Nos. 9,670,183; 9,926,297; 10,196,380; 10,385,039; 10,689,368; and 10,961,225; the disclosures of which are each incorporated herein by reference in their entireties.

In some embodiments, the present application provides methods of treating a disease or disorder associated with muscarinic M4 receptor. In some embodiments, the method comprises administering to a subject (e.g., a subject in need thereof), a compound provided herein (e.g., a therapeutically effective amount of a compound provided herein), or a pharmaceutically acceptable salt thereof.

In some embodiments, the present application provides a method of treating a cognitive disorder, a psychotic disorder, a movement disorder, addiction, or for treating or lessening the severity of acute, chronic, neuropathic, or inflammatory pain in a subject in need thereof, comprising administering to the subject a compound provided herein, or a pharmaceutically acceptable salt thereof.

In some embodiments, the present application provides a method of treating a cognitive disorder or psychotic disorder in a subject in need thereof.

In some embodiments, the cognitive disorder or psychotic disorder comprises, arises from, or is associated with a condition selected from cognitive impairment, Mild Cognitive Impairment, frontotemporal dementia, vascular dementia, dementia with Lewy bodies, presenile dementia, senile dementia, Friederich's ataxia, Down's syndrome, Huntington's chorea, hyperkinesia, mania, Tourette's syndrome, Alzheimer's disease, progressive supranuclear palsy, impairment of cognitive functions including attention, orientation, learning disorders, memory (i.e., memory disorders, amnesia, amnesic disorders, transient global amnesia syndrome and age-associated memory impairment) and language function; cognitive impairment as a result of stroke, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, age related cognitive impairment, autism related cognitive impairment, Down's syndrome, cognitive deficit related to psychosis (such as dementia related psychosis), and post-electroconvulsive treatment related cognitive disorders; cognitive disorders due to drug abuse or drug withdrawal including nicotine, cannabis, amphetamine, cocaine, Attention Deficit Hyperactivity Disorder (ADHD) and dyskinetic disorders such as Parkinson's disease, neuroleptic-induced parkinsonism, and tardive dyskinesias, schizophrenia, schizophreniform diseases, psychotic depression, mania, acute mania, paranoid, hallucinogenic and delusional disorders, personality disorders, obsessive compulsive disorders, schizotypal disorders, delusional disorders, psychosis due to malignancy, metabolic disorder, endocrine disease or narcolepsy, psychosis due to drug abuse or drug withdrawal, bipolar disorders, epilepsy, and schizo-affective disorder.

In some embodiments, the psychotic disorder is schizophrenia.

In some embodiments, the present application provides a method of treating Alzheimer's Disease or dementia with Lewy bodies in a subject in need thereof, comprising administering to the subject a compound provided herein, or a pharmaceutically acceptable salt thereof. In some embodiments, the method is a method of treating Alzheimer's Disease. In some embodiments, the method is a method of treating dementia with Lewy bodies.

In some embodiments, the present application provides a method of treating or lessening the severity of acute, chronic, neuropathic, or inflammatory pain, arthritis, migraine, cluster headaches, trigeminal neuralgia, herpetic neuralgia, general neuralgias, visceral pain, osteoarthritis pain, postherpetic neuralgia, diabetic neuropathy, radicular pain, sciatica, back pain, head or neck pain, severe or intractable pain, nociceptive pain, breakthrough pain, postsurgical pain, or cancer pain, in a subject in need thereof, comprising administering to the subject a compound provided herein, or a pharmaceutically acceptable salt thereof.

In some embodiments, the present application provides a method of treating peripheral disorders such as reduction of intra ocular pressure in glaucoma, in a subject in need thereof, comprising administering to the subject a compound provided herein, or a pharmaceutically acceptable salt thereof.

In some embodiments, the present application provides a method of treating dry eye, in a subject in need thereof, comprising administering to the subject a compound provided herein, or a pharmaceutically acceptable salt thereof.

In some embodiments, the present application provides a method of treating dry mouth (including Sjogren's Syndrome) in a subject in need thereof, comprising administering to the subject a compound provided herein, or a pharmaceutically acceptable salt thereof.

In some embodiments, the present application provides a method of treating addiction in a subject in need thereof, comprising administering to the subject a compound provided herein, or a pharmaceutically acceptable salt thereof.

In some embodiments, the present application provides a method of treating a movement disorder in a subject in need thereof, comprising administering to the subject a compound provided herein, or a pharmaceutically acceptable salt thereof. In some embodiments, the movement disorder is selected from such as Parkinson's disease, ADHD, Huntingdon's disease, tourette's syndrome, and other syndromes associated with dopaminergic dysfunction as an underlying pathogenetic factor driving disease.

As used herein, the term “contacting” refers to the bringing together of indicated moieties in an in vitro system or an in vivo system.

As used herein, the term “patient” or “subject” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, subject, or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In some embodiments, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in a subject who is experiencing or displaying the pathology or symptomatology of the disease, condition or disorder (i.e., arresting further development of the pathology and/or symptomatology) or ameliorating the disease; for example, ameliorating a disease, condition or disorder in a subject who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in a subject who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Pharmaceutical Formulations

The present application further provides pharmaceutical compositions containing the compounds provided herein. In some embodiments, the present application provides a composition (e.g., a pharmaceutical composition) comprising a compound provided herein (e.g., a compound of any of Formulas I-IVa), or a pharmaceutically acceptable salt thereof, in a pharmaceutically acceptable carrier, excipient or diluent.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term “parenteral” as used herein includes subcutaneous injections, intravenous, intramuscular, intrasternal injection, or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; see e.g., The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions provided herein further comprise a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like.

A thorough discussion of pharmaceutically acceptable carriers, diluents, and other excipients is presented in Remington's Pharmaceutical Sciences (Mack Pub. Co., N.J. current edition).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The most suitable route will depend on the nature and severity of the condition being treated. Those skilled in the art are also familiar with determining administration methods (e.g., oral, intravenous, inhalation, sub-cutaneous, rectal, etc.), dosage forms, suitable pharmaceutical excipients and other matters relevant to the delivery of the compounds to a subject in need thereof.

### Kits

The present invention also provides kits that contain a pharmaceutical composition which includes one or more compounds of the invention. The kit also includes instructions for the use of the pharmaceutical composition for the treatment of any of the disease and disorders described herein, as well as other utilities as disclosed herein. In some embodiments, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. In Vitro Metabolite Profile and Metabolite Identification of Compound 1

Metabolite profile and metabolite identification of Compound 1 were studied in cryopreserved SD-rat, beagle dog and human hepatocytes. The incubations were conducted using 10 μM initial concentrations of test substance and sampling at 0 min, 60 min, and 120 min. The samples were analysed using UPLC/Q-TOF-MS. Incubation material and procedures are provided Table 1. The samples were thawed at room temperature (RT), centrifuged (10 min at 13000×g (Heraeus Pico 17 centrifuge), and the supernatants were pipetted to glass vials to wait for analysis. Analysis was performed using liquid chromatography-mass spectrometry (LC-MS) according to the parameters described in Table 2. The samples were thawed at room temperature (RT), centrifuged (10 min at 13000×g (Heraeus Pico 17 centrifuge), and the supernatants were pipetted to glass vials to wait for analysis.

TABLE 1

| | |
|---|---|
| Enzyme source | Pooled cryopreserved hepatocytes, Celsis IVT, |
| Species/strain | Human/mixed gender |
| | Dog/male beagle |
| | Rat/male SD |

Conditions in the final incubation:

| Incubation volume | 300 μL in 48-well plate | |
|---|---|---|
| Cell viability, determined using | 1.0 million viable cells/mL | |
| Trypan blue-method | | |
| Test compound | 10 μM (stock solution in 50% acetonitrile:water) | |
| Solvent content in incubation | 0.25% (acetonitrile) | |
| Other conditions | pH 7.4, BioreclamationITV In Vitro GRO HI | |
| | medium | |
| Shaking | 600 | rpm |
| Time points | 0, 60, and 120 min with and without cells + | |
| | 120 min blank (without compound) | |
| Temperature | 37° | C. |
| Sampling volume | 60 | μL |

| | |
|---|---|
| Termination of incubations | Equal volume of acetonitrile |
| Storage | −20° C. until analysis |
| Disapperance control | verapamil |

TABLE 2

| | |
|---|---|
| Instrumentation | Waters Acquity UPLC + Acquity PDA-detector + |
| | Waters Xevo G2 Q-TOF-MS |
| Column | Waters Acquity BEH C18 (2.1 × 50 mm, |
| | 1.8 μm) column with guard filter |
| Polarity | ESI+ |
| Desolvatation Gas | nitrogen 900 (L/h) |
| Cone Gas | nitrogen 50 (L/h) |
| Capillary voltage | 500 V |
| Cone voltage | 30 V |
| Desolvation Temp | 650 (° C.) |
| Source Temp | 150 (° C.) |
| DRE | OFF |
| Ion optics | Resolution |
| Mass range | m/z 100-1200 |
| Acquisition time | 100 ms |
| Aperture voltage (MSe) | 3 eV (parent ion function), 25-50 eV |
| | (fragment ion function) |
| Resolution | 24 000 (FWHM) |
| Lock mass compound | Leucine encephalin MH+ m/z 556.2771 |
| UV-range | 210-500 nm |
| Software | Masslynx 4.1 (including MetaboLynx XE) |
| Other information | First 0.5 min of the run was directed into waste by using a divert |
| | valve to decrease the ion source contamination by early eluting |
| | matrix constituents. |

Gradient Elution; A = 0.05% ammonia, B = acetonitrile

| Time | Flow | | A % | B % | curve |
|---|---|---|---|---|---|
| 0.00 | 0.500 | mL/min | 98 | 2 | — |
| 1.00 | 0.500 | mL/min | 98 | 2 | 6 |
| 3.50 | 0.500 | mL/min | 60 | 40 | 6 |
| 4.00 | 0.500 | mL/min | 10 | 90 | 6 |
| 5.00 | 0.500 | mL/min | 98 | 2 | 1 |
| Temperature | 35 | (° C.) | | | |
| Injection Volume | 4 | (μL) | | | |

| | |
|---|---|
| Data processing | Ion chromatograms were extracted from the TOF-MS total ion |
| | chromatograms using calculated monoisotopic accurate masses (calculated |
| | using Waters Masslynx software for deprotonated molecule) with 15 mDa |
| | window. The metabolites (expected + unexpected) were mined from the |
| | data acquired from the last time point, using software-aided data |
| | processing (Metabolynx XS including structure-intelligent dealkylation |
| | tool & mass defect filter) with manual confirmation. Structures of the |
| | observed metabolites are tentatively identified using obtained accurate |
| | mass and fragment ion data. |

Compound 1 showed only low metabolic turnover in incubation with hepatocytes; the remaining abundances after 120 min incubation period in all species were 79-93%. Practically no disappearance was observed in the control incubation without cells. Results for the positive control compound verapamil showed that enzyme activities were at an acceptable level.

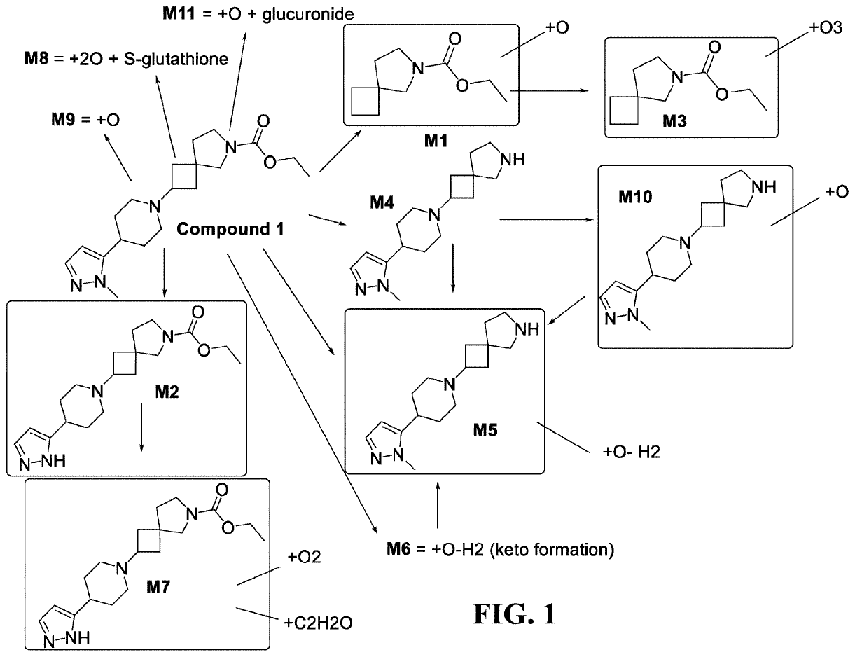

Metabolite profiles in incubations with hepatocytes are provided in Table 3. In total eleven metabolites (M1-M11) were observed for Compound 1. The metabolites were formed mainly via various dealkylation reactions and their further hydroxylations; i.e., loss of C₉H₁₃N₃ (in M1 and M3), N-demethylation (in M2, M5, M7), and loss of C₃H₄O₂ (in M4, M5, M10). Also, hydroxylation/oxidation without dealkylation was observed (M9), as well as with dehydrogenation (keto-formation, M6), and with S-glutathione and glucuronide-conjugations (M8, M11). Acetylation was observed in M7. Eight of these metabolites (M1-M8) were observed with human hepatocytes. Minor metabolite M8 was specific to human only. The M₄ appeared to be the major metabolite with rat and dog hepatocytes. The proposed metabolic pathways for the observed Compound 1 metabolites are shown in FIG. 1.

TABLE 3

| | Human | Rat | Dog | No cells |
|---|---|---|---|---|
| Compound | 120 min, % | 120 min, % | 120 min, % | 120 min, % |
| Compound 1 | 84.0 | 93.7 | 94.9 | 100 |
| M1 | 1.1 | — | 0.09 | — |
| M2 | 0.3 | 0.1 | — | — |
| M3 | 0.1 | 0.3 | 0.1 | — |
| M4 | 13.8 | 4.9 | 4.6 | — |
| M5 | 0.2 | 0.5 | — | — |
| M6 | 0.2 | 0.1 | 0.1 | — |
| M7 | 0.1 | — | 0.1 | — |
| M8 | 0.2 | — | — | — |
| M9 | — | 0.2 | — | — |
| M10 | — | 0.1 | 0.1 | — |
| M11 | — | 0.2 | — | — |

The metabolism of Compound 1 was qualitatively similar in all species. The two primary pathways identified in human hepatocytes were:
1. Decarboethoxylation to form metabolite M4
2. N-demethylation to form metabolite M2

Proposed structures of these metabolites are shown below.

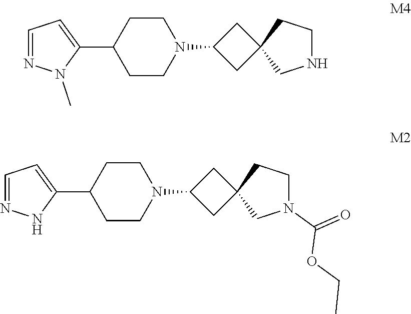 [SMILES: Cn1nccc1C1CCN([C@H]2C[C@]3(CCNC3)C2)CC1 ; CCOC(=O)N1CC[C@]2(C1)C[C@@H](N1CCC(c3ccn[nH]3)CC1)C2]

Metabolites M4 and M2 are formed via oxidative and/or hydrolytic mechanisms with no direct Phase 2 conjugation of parent Compound 1 observed. Both metabolites were observed in all species evaluated supporting the use of these species for the toxicological evaluation of Compound 1. A number of other metabolites were observed at trace levels. The metabolic pathways involved in the formation of these minor metabolites include oxidation, desaturation, dealkylation, and secondary Phase 2 conjugation (acetylation, formylation, glucuronidation, glycosylation, and glutathione conjugation).

### Example 2. In Vivo Metabolite Profile and Metabolite Identification of Compound 1

The in vivo metabolism of Compound 1 was investigated in studies using rat, dog, rabbit, and human plasma and human urine samples from toxicology studies, an embryofetal development study, and a human clinical study.

### Human Samples

Human plasma and urine samples originated from a human clinical study with subjects aged 65 years or older, dosed with 15 mg daily and achieving steady-state before day 7. Samples were stored at −75° C. or below from collection until analysis.

Human plasma samples from each Compound 1-dosed subject (15 mg; n=8) collected at day 7 at 0.25 h, 0.5 h, 1 h, 1.5 h, 2 h, 3 h, 4 h, 6 h, 8 h, 10 h, 12 h and 24 h after multiple daily oral administration were pooled for analysis. Plasma from placebo-dosed subjects (n=2) taken at the same time-points and pre-dose plasma collected on day 1 from Compound 1-dosed subjects were used as control for metabolite identification.

Human urine samples from the Compound 1-dosed subjects (15 mg; n=8) collected at day 7 between 0-4 h, 4 h-8 h, 8 h-12 h and 12 h-24 h after multiple daily oral administration were also pooled for analysis. Urine from placebo-dosed subjects (n=2) collected at day 7 over the same time periods was used as control for metabolite identification.

### Pooling of Plasma Samples and Preparation for Metabolite Identification

Time-normalized pooled plasma samples (day 7; 0-24 h) were prepared for each human subject treated with Compound 1 (15 mg; n=8) or with placebo (n=2) (see e.g., Hamilton et al, Clin. Pharmacol. Ther. 1983, 29(3):408-13). Composite time-normalized pooled plasma samples for human subjects dosed with Compound 1 or placebo were then prepared by combining an equal volume from the pooled sample prepared for each subject. A composite control plasma sample was prepared by combining an equal volume of pre-dose plasma from human subjects treated with Compound 1 (n=8).

Aliquots (100 μL) of the pooled plasma samples prepared for metabolite identification were treated with ice-cold acetonitrile (500 μL), mixed and centrifuged (2000 g; 4° C.; 10 min). The supernatants (550 μL) were removed and reduced to dryness under a stream of nitrogen (35° C.) and the dry extracts reconstituted in 200 μL 90:10 (v/v) water:acetonitrile prior to analysis. Surplus sample aliquots were stored at −80° C.

### Pooling of Human Urine Samples and Preparation for Metabolite Identification

A composite urine sample (day 7; 0-24 h) was prepared for each human subject treated with Compound 1 (15 mg; n=8) or with placebo (n=2) by combining proportional amounts of the sample collected over each time period. Composite cross-subject urine samples for human subjects dosed with Compound 1 or placebo were then prepared by combining proportional amounts of pooled urine from each subject. All urine pools were mixed and centrifuged (2000 g; 4° C.; 10 min) prior to analysis. Surplus sample aliquots were stored at −80° C.

### LC-MS Identification of the Metabolites of Compound 1

Samples were analysed by LC-MS, utilising a Vion Q-TOF mass spectrometer (Waters), Acquity I-class LC pumps, column heater, sample manager and photodiode array detector (Waters). The mass spectrometer was set to operate in positive ion mode and a number of experiments were performed to enable the acquisition of full scan and product ion data. Metabolite “identification” refers to putative metabolites deemed to be related to Compound 1 by comparison of their predicted and observed accurate mass and other appropriate LC-MS techniques. Metabolite abundance was calculated as a percentage of total drug-related material in a given sample; the MS peak areas were collated and summed, and the abundance of each metabolite calculated as a percentage of the total in each sample. Based on apparent abundance, metabolites were categorized as ‘major’ if present at 10% or more of the total drug-related material, ‘minor’ if between 1 and <10% and ‘trace’ if <1%.

### HPLC Conditions

| | |
|---|---|
| Column | Xselect CSH, 150 × 2.1 mm, 2.5 μm, C18, XP (Waters) |
| Solvent A | 0.2% ammonia in water |
| Solvent B | acetonitrile/methanol/water (0.45:0.45:0.1, v/v/v) |
| | containing 0.4% formic acid |
| Flow rate | 500 μL/min |
| Temperature | 50° C. |
| Gradient | 0.0 min 10% B |
| | 15.0 min 35% B |
| | 17.0 min 95% B |
| | 20.0 min 95% B |
| | 20.1 min 10% B |
| | 30.0 min 10% B |

MS Conditions

| Polarity | Positive |
|---|---|
| Resolution | >25000 |
| Source Temp (° C.) | 150 |
| Capillary Voltage (V) | 1100 |
| Desolvation Temperature (° C.) | 500 |
| Desolvation gas (L/h) | 1000 |
| Cone gas (L/h) | 100 |
| Cone voltage (V) | 40 |

Identification of the Metabolites of Compound 1 in Human Plasma from a Cross-Subject Pool

LC-MS analysis of cross-subject human plasma (n=8; day 7, 0-24 h pool) indicated that unchanged Compound 1 (m/z 347; P [parent]) was the predominant drug-related component in cross-subject human plasma, accounting for 82.0% of the total drug-related material. A major product resulting from hydrolysis of the ethyl carbamate moiety (m/z 275; P-72; metabolite M4; the product of decarboethoxylation) appeared to be the most abundant metabolite in cross-subject human plasma and accounted for 11.4% of the total drug-related material. A product of mono-oxidation of the ethyl group of the ethyl carbamate moiety (m/z 363; P+16; metabolite M7) and a product of N-demethylation (m/z 333; P-14; metabolite M2) were detected as minor metabolites, accounting for 1.0% and 2.3% respectively of the total drug-related material. Several trace metabolites (each estimated to account for <1% of the total drug-related material) were detected in human plasma: a product consistent with hydrolysis of the ethyl carbamate moiety in combination with glucose conjugation (m/z 437; P+90M4+glucose), a product postulated to result from hydrolysis of the ethyl carbamate moiety in combination with formylation (m/z 303; P-44; metabolite M5), a product of hydration (i.e., addition of H₂O) in the azaspiro moiety (m/z 365; P+18; metabolite M8), a product of mono-oxidation in the azaspiro moiety (m/z 363; P+16; metabolite M10), a product of mono-oxidation in either the azaspiro, piperidine or pyrazole ring (m/z 363; P+16; metabolite M11), a product of mono-oxidation in either the piperidine or pyrazole ring (m/z 363; P+16; metabolite M13), a product postulated to result from hydrolysis of the ethyl carbamate moiety in combination with acetylation (m/z 317; P-30; metabolite M14), a product resulting from N-demethylation in combination with mono-oxidation (m/z 349, P+2; metabolite M17), a product consistent with hydrolysis of the ethyl carbamate moiety in combination with N-demethylation (m/z 261, P-86; metabolite M18) and a product of mono-oxidation (m/z 363; P+16; metabolite M19). Additional low abundance secondary metabolites involving hydrolysis of the ethyl carbamate group and/or oxidation were also detected.

Identification of the Metabolites of Compound 1 in Human Urine from a Cross-Subject Pool

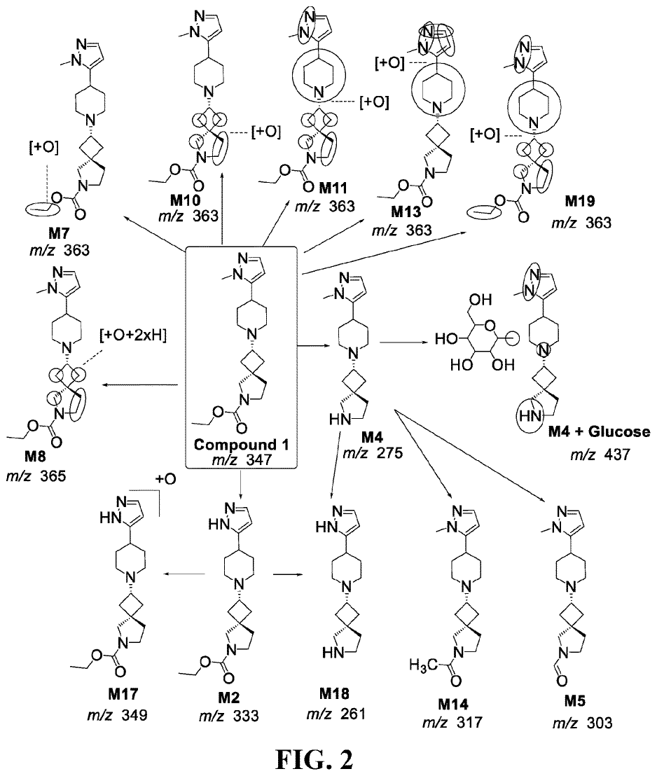

LC-MS analysis of cross-subject human urine (n=8; day 7, 0-24 h pool) indicated that unchanged Compound 1 (m/z 347; P [parent]) was the predominant drug-related component in cross-subject human urine, accounting for 63.3% of the total drug-related material. A product resulting from hydrolysis of the ethyl carbamate moiety (m/z 275; P-72; metabolite M4) appeared to be a major metabolite in cross-subject human urine and accounted for 26.4% of the total drug-related material. Several minor metabolites (estimated to account for between 1.2 and 3.3% of the total drug-related material) were observed in human urine: a product of mono-oxidation of the ethyl group of the ethyl carbamate moiety (m/z 363; P+16; metabolite M7), a product of hydration (i.e., addition of H₂O) in the azaspiro moiety (m/z 365; P+18; metabolite M8), a product of N-demethylation (m/z 333; P-14; metabolite M2) and a product consistent with hydrolysis of the ethyl carbamate moiety in combination with N-demethylation (m/z 261; P-86; metabolite M18). Several trace metabolites (each estimated to account for <1% of the total drug-related material) were also detected in human urine: a product postulated to result from hydrolysis of the ethyl carbamate moiety in combination with formylation (m/z 303; P-44; metabolite M5), a product of mono-oxidation in the azaspiro moiety (m/z 363; P+16; metabolite M10), a product of mono-oxidation in either the azaspiro, piperidine or pyrazole ring (m/z 363; P+16; metabolite M11), a product postulated to result from hydrolysis of the ethyl carbamate moiety in combination with acetylation (m/z 317; P-30; metabolite M14), a product resulting from N-demethylation in combination with mono-oxidation (m/z 349, P+2; metabolite M17) and a product of mono-oxidation (m/z 363; P+16; metabolite M19). A summary of proposed metabolites of Compound 1 in human plasma and urine following multiple daily oral administration is provided in Table 4 and FIG. 2.

TABLE 4

| | | | | % Abundance in | |
|---|---|---|---|---|---|
| | | | | Sample | |
| | | | | Multiple 15 mg | |
| | | | RT^{a} | Administration | |
| M# | Assignment | m/z | (min) | Plasma^{b} | Urine^{c} |
| P | Compound 1 | 347 | 10.3 | 82.0 | 63.3 |
| M2 | N-demethylation | 333 | 10.1 | 2.3 | 3.3 |
| M4 | Ethyl carbamate hydrolysis | 275 | 8.1 | 11.4 | 26.4 |
| M4 + glucose | Ethyl carbamate hydrolysis, | 437 | 8.0 | 0.3 | ND |
| | glucose conjugation | | | | |
| M5 | Ethyl carbamate hydrolysis, | 303 | 8.1 | 0.3 | <0.1 |
| | formylation | | | | |
| M7 | Mono-oxidation (ethyl carbamate) | 363 | 8.2 | 1.0 | 2.1 |
| M8 | Hydration (azaspiro) | 365 | 8.4 | 0.5 | 1.2 |
| M10 | Mono-oxidation (azaspiro) | 363 | 8.6 | 0.3 | 0.5 |
| M11 | Mono-oxidation | 363 | 9.5 | <0.1 | 0.1 |
| | (azaspiro/piperidine/pyrazole) | | | | |
| M13 | Mono-oxidation | 363 | 10.4 | 0.5 | ND |
| | (piperidine/pyrazole) | | | | |
| M14 | Ethyl carbamate hydrolysis, | 317 | 8.1 | 0.3 | 0.8 |
| | acetylation | | | | |
| M17 | Mono-oxidation, N-demethylation | 349 | 8.1 | 0.3 | 0.6 |
| M18 | Ethyl carbamate hydrolysis, | 261 | 8.1 | 0.6 | 1.3 |
| | N-demethylation | | | | |
| M19 | Mono-oxidation | 363 | 8.7 | 0.2 | 0.3 |

^{a}Values taken from human plasma
^{b}Composite time-normalized human plasma sample (day 7; 0-24 h) from Compound 1-dosed subjects
^{c}Composite human urine sample (day 7; 0-24 h) from Compound 1-dosed subjects
ND = Not Detected

Analysis of rat, dog, rabbit, and human plasma samples showed unchanged Compound 1 to be the predominant circulating component. Consistent with in vitro data (Example 1), metabolites M4 and M2 were the two most abundant human circulating metabolites of Compound 1, representing an estimated 11.4% (M4) and 2.3% (M2), of total drug-related material in plasma collected following multiple oral 15 mg doses of Compound 1. These metabolites were also observed in rat, dog, and rabbit plasma. Other human circulating metabolites were observed at trace levels.

In general, metabolites detected in human urine were qualitatively similar to that in human plasma. In urine samples collected following multiple 15 mg oral doses of Compound 1, parent drug was the predominant drug-related component, accounting for an estimated 63.3% of total drug-related material. Metabolite M4 was identified as the most abundant metabolite in human urine and accounted for 26.4% of the total drug-related material.

A preliminary comparison of Compound 1 metabolite exposures in rat, dog, and rabbit plasma relative to human was also performed. Pooled rat and dog plasma from 13-week toxicology studies, pooled rabbit plasma from an embryofetal development study, and pooled human plasma from a clinical study were analyzed by LC-MS/MS. The exposures of metabolites in pooled animal plasma samples were compared to those in the human pool by comparing the relative LC-MS-MS peak areas. Metabolites evaluated included metabolite M4, metabolite M2, and additional trace-level human metabolites. The relative abundance of metabolites measured using LC-MS/MS was greater in the plasma of rat, rabbit, dog, and monkey than in humans. These data suggest that the safety of these human metabolites has been adequately assessed in the nonclinical species.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I: 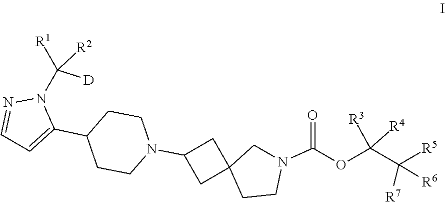
or a pharmaceutically acceptable salt thereof,
wherein R¹, R², R³, R⁴, R⁵, R⁶, and R⁷ are each independently selected from hydrogen and deuterium.

2. The compound of claim 1, or a pharmaceutically acceptable salt thereof, wherein one of R¹, R², R³, R⁴, R⁵, R⁶, and R⁷ is deuterium.

3. The compound of claim 1, or a pharmaceutically acceptable salt thereof, wherein two of R¹, R², R³, R⁴, R⁵, R⁶, and R⁷ are each deuterium.

4. The compound of claim 1, or a pharmaceutically acceptable salt thereof, wherein three of R¹, R², R³, R⁴, R⁵, R⁶, and R⁷ are each deuterium.

5. The compound of claim 1, or a pharmaceutically acceptable salt thereof, wherein four of R¹, R², R³, R⁴, R⁵, R⁶, and R⁷ are each deuterium.

6. The compound of claim 1, or a pharmaceutically acceptable salt thereof, wherein five of R¹, R², R³, R⁴, R⁵, R⁶, and R⁷ are each deuterium.

7. The compound of claim 1, or a pharmaceutically acceptable salt thereof, wherein R¹, R², R³, R⁴, R⁵, R⁶, and R⁷ are each deuterium.

8. The compound of claim 1, which is a compound of Formula II: 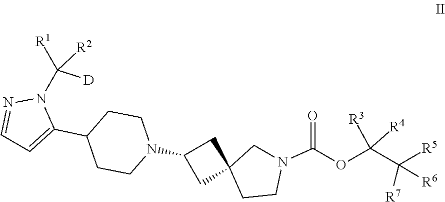
or a pharmaceutically acceptable salt thereof.

9. The compound of claim 1, which is a compound of Formula III: 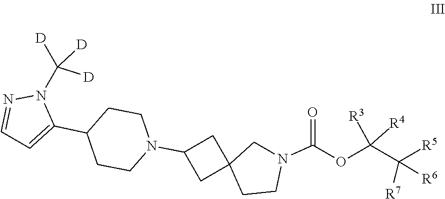
or a pharmaceutically acceptable salt thereof.

10. The compound of claim 1, which is a compound of Formula IIIa: 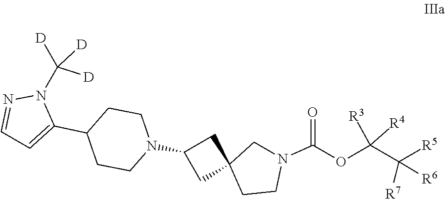
or a pharmaceutically acceptable salt thereof.

11. The compound of claim 1, which is a compound of Formula IV: 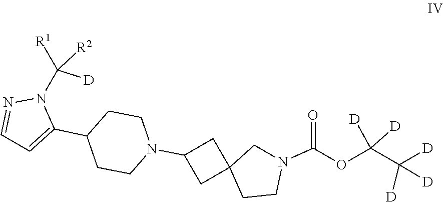
or a pharmaceutically acceptable salt thereof.

12. The compound of claim 1, which is a compound of Formula IVa: 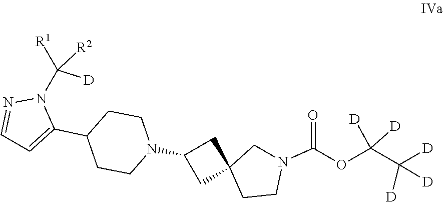
or a pharmaceutically acceptable salt thereof.

13. The compound of claim 1, which is selected from: 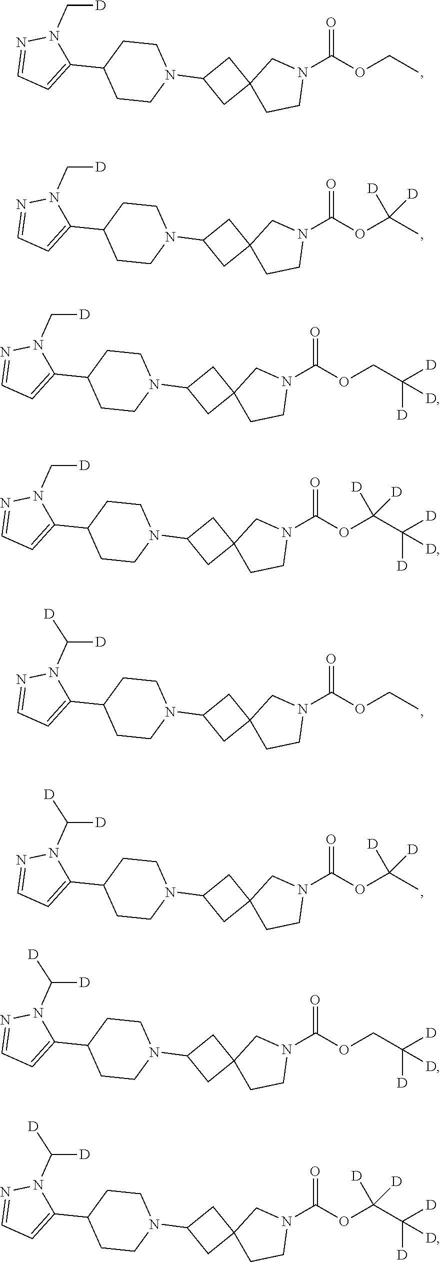 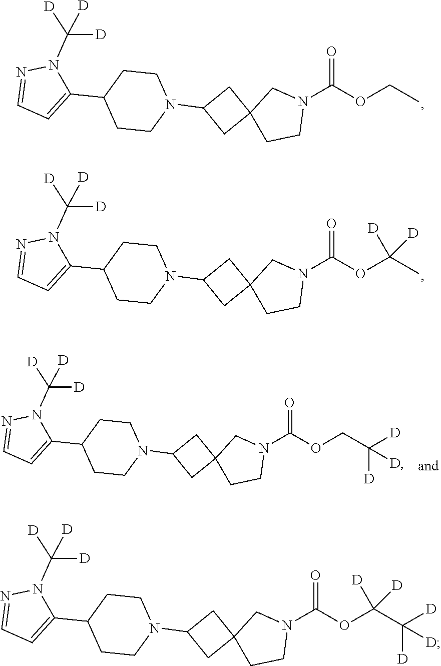
or a pharmaceutically acceptable salt thereof.

14. The compound of claim 1, which is selected from: 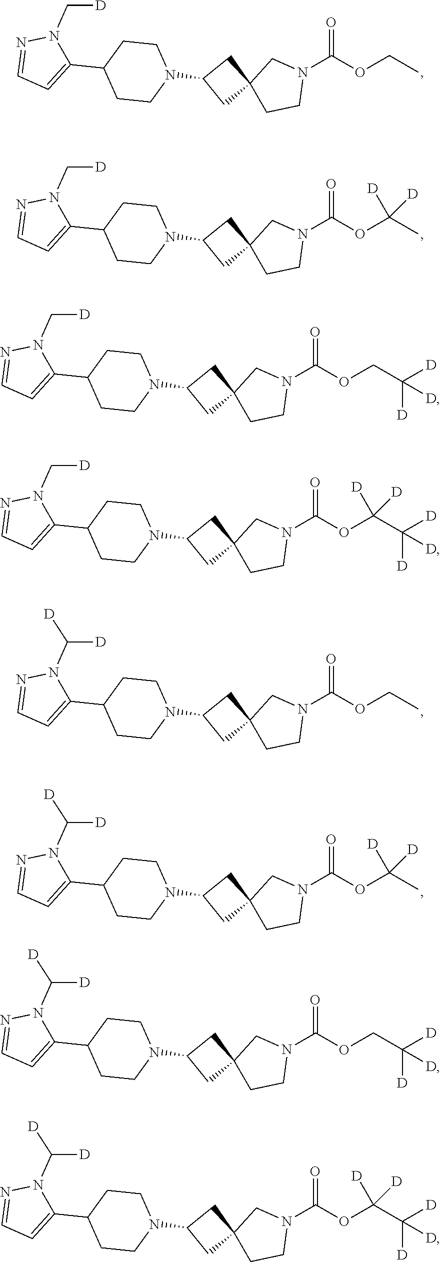 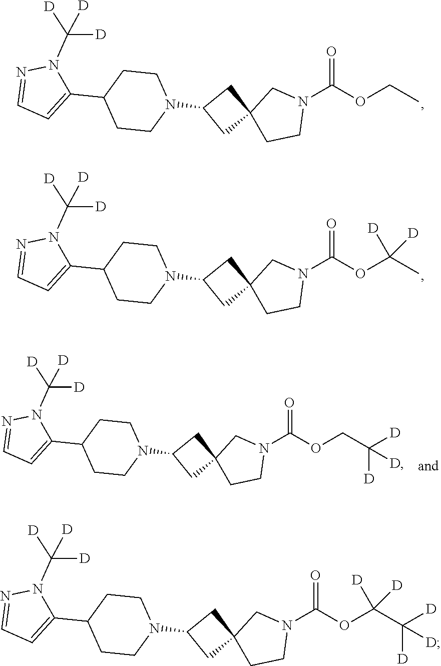
or a pharmaceutically acceptable salt thereof.

15. A pharmaceutical composition comprising a compound of any one of claims 1 to 14, or a pharmaceutically acceptable salt thereof, and a pharmaceutically acceptable carrier.

16. A method of treating a cognitive disorder, a psychotic disorder, a movement disorder, addiction, or for treating or lessening the severity of acute, chronic, neuropathic, or inflammatory pain in a subject in need thereof, comprising administering to the subject a compound of any one of claims 1 to 14, or a pharmaceutically acceptable salt thereof.

17. The method of claim 16, wherein the psychotic disorder is schizophrenia.

18. A method of treating Alzheimer's Disease or dementia with Lewy bodies in a subject in need thereof, comprising administering to the subject a compound of any one of claims 1 to 14, or a pharmaceutically acceptable salt thereof.

19. The method of claim 18, which is a method of treating Alzheimer's Disease.

20. The method of claim 18, which is a method of treating dementia with Lewy bodies.
